(19) United States
(12) Patent Application Publication
(10) Pub. No.: US 2025/0099782 A1
(43) Pub. Date: Mar. 27, 2025

(54) Method for Generating Radiation Therapy Plan and Electronic Device

(71) Applicant: OUR UNITED CORPORATION, Shaanxi (CN)

(72) Inventors: Yipeng Jing, Shaanxi (CN); Xikang Zhao, Shaanxi (CN)

(21) Appl. No.: 18/385,593

(22) Filed: Oct. 31, 2023

(30) Foreign Application Priority Data
Sep. 27, 2023 (CN) 202311267880.4

(51) Int. Cl. A61N 5/10

(52) U.S. Cl. CPC A61N 5/103; A61N 2005/1041

(57) ABSTRACT

A method for generating a radiation therapy plan is provided. The method includes acquiring a first radiation therapy plan for an object that is to be treated with radiation therapy in a first radiation therapy mode; in response to a dose conversion operation on the first radiation therapy plan, acquiring a first effective dose field obtained by dose conversion of the first radiation therapy plan, the first effective dose field being used to evaluate effect of radiation therapy based on the first radiation therapy plan on different radiation volumes in the object; and generating a second radiation therapy plan for the object in a second radiation therapy mode using the first effective dose field as a basic dose field.

### CROSS-REFERENCE TO RELATED APPLICATION

This application claims priority to Chinese Patent Application No. 202311267880.4, filed Sep. 27, 2023, the disclosure of which is hereby incorporated by reference in its entirety.

### BACKGROUND OF THE INVENTION

### Field of the Invention

The present disclosure relates to the field of medical technology, and in particular, to a method for generating a radiation therapy plan and an electronic device.

### Description of Related Art

With the rapid development of computer technology and imaging technology, the technology of radiation therapy (also called radiotherapy, RT for short) has been maturing. The radiation therapy, which is one of the important means for treating tumors, achieves the purpose of destroying a tumor by giving the tumor a sufficiently high radiation dose.

In order to ensure that normal tissue surrounding a tumor is not irradiated by too high dose while a high dose is given to the tumor, a corresponding radiation therapy plan is usually developed based on a prescription dose for an object to be treated with radiation therapy (i.e., a patient). In addition, in the process of developing the radiation therapy plan, the developed radiation therapy plan will be further evaluated to generate a radiation therapy plan with a good therapeutic effect.

In particular, in a case where an object to be treated with radiation therapy corresponds to different radiation therapy modes, how to generate a radiation therapy plan for the object to be treated with radiation therapy is a matter of interest.

### SUMMARY OF THE INVENTION

The present disclosure provides a method for generating a radiation therapy plan and an electronic device.

According to an aspect of the present disclosure, a method for generating a radiation therapy plan is provided, which includes:
acquiring a first radiation therapy plan for an object that is to be treated with radiation therapy in a first radiation therapy mode;
in response to a dose conversion operation on the first radiation therapy plan, acquiring a first effective dose field obtained by dose conversion of the first radiation therapy plan, wherein the first effective dose field is used to evaluate effect of radiation therapy based on the first radiation therapy plan on different radiation volumes in the object; and
generating a second radiation therapy plan for the object in a second radiation therapy mode using the first effective dose field as a basic dose field.

According to another aspect of the present disclosure, another method for generating a radiation therapy plan is provided, which includes:
acquiring a first radiation therapy plan for an object that is to be treated with radiation therapy in a first radiation therapy mode;
in response to a dose conversion operation on the first radiation therapy plan, acquiring a first effective dose field obtained by dose conversion of the first radiation therapy plan, wherein the first effective dose field is used to evaluate effect of radiation therapy based on the first radiation therapy plan on different radiation volumes in the object;
evaluating whether an original dose field of the first radiation therapy plan and the first effective dose field satisfy a first prescription condition; and
generating a second radiation therapy plan for the object in a second radiation therapy mode using one, satisfying the first prescription condition, of the original dose field and the first effective dose field as the basic dose field, based on an evaluation result of the evaluating.

According to yet another aspect of the present disclosure, an electronic device is provided, which includes: at least one processor, and a memory communicatively connected to the at least one processor. The memory stores instructions executable by the at least one processor; and the instructions, when executed by the at least one processor, cause the at least one processor to perform any method for generating a radiation therapy plan provided by the present disclosure.

According to still yet another aspect of the present disclosure, a non-transitory computer-readable storage medium is provided. The non-transitory computer-readable storage medium has stored computer instructions that, when run on an electronic device, cause the electronic device to perform any method for generating a radiation therapy plan provided by the present disclosure.

According to still yet another aspect of the present disclosure, a computer program product is provided, including a computer program that, when executed by a processor, implements any method for generating a radiation therapy plan provided by the present disclosure.

It should be understood that what is described in the summary is not intended to identify critical or significant features of the embodiments of the disclosure, nor is it intended to limit the scope of the present disclosure. Other features of the present disclosure will become readily understood from the following description.

### BRIEF DESCRIPTION OF THE DRAWINGS

The accompanying drawings are used for a fuller understanding of the present solution and do not constitute a limitation of the present disclosure, in which:

FIG. 1 is a schematic diagram showing an implementation environment of a method for generating a radiation therapy plan, in accordance with some embodiments of the present disclosure;

FIG. 2A is a schematic flowchart showing a method for generating a radiation therapy plan, in accordance with some embodiments of the present disclosure;

FIG. 2B is a schematic flowchart showing a method for generating another radiation therapy plan, in accordance with some embodiments of the present disclosure;

FIG. 3 is a schematic flowchart showing a method for generating a radiation therapy plan, in accordance with some embodiments of the present disclosure;

FIG. 4 is a block diagram showing an electronic device, in accordance with some embodiments; and

FIG. 5 is another block diagram showing an electronic device, in accordance with some embodiments.

### DESCRIPTION OF THE INVENTION

Exemplary embodiments of the present disclosure are described below with reference to the accompanying drawings, in which various details of the embodiments of the present disclosure are included to facilitate understanding and should be considered to be exemplary only. Accordingly, those of ordinary skill in the art will recognize that various changes and modifications can be made to the embodiments described herein without departing from the scope and spirit of the present disclosure. Also, descriptions of well-known functions and constructions are omitted from the following description for clarity and conciseness.

As used herein, “and/or” is merely an association relationship for describing associated objects, which represents that there may be three kinds of relationships. For example, “A and/or B” may represent three situations that: A exists only, A and B exist simultaneously, and B exists only. In addition, “at least one” means one or more; “a/the of plurality” means two or more.

As used herein, the term “if” is optionally construed as “when”, “in a case where”, “in response to determining” or “in response to detecting”, depending on the context. Similarly, depending on the context, the phrase “if it is determined that” or “if [a stated condition or event] is detected” is optionally construed as “in a case where it is determined that”, or “in response to determining that”, or “in a case where [the stated condition or event] is detected” or “in response to detecting [the stated condition or event]”.

In addition, the use of the phrase “based on” is meant to be open and inclusive, since a process, step, calculation or other action that is “based on” one or more of the stated conditions or values may, in practice, be based on additional conditions or values beyond those stated.

First, application scenarios involved in the embodiments of the present disclosure are described. The method for generating a radiation therapy plan provided by the embodiments of the present disclosure may be applied in the field of medical technology, and specifically may be applied in radiation therapy scenarios.

With the rapid development of computer technology and imaging technology, radiation therapy techniques have also been maturing, such as three-dimensional conformal radiation therapy (3D-CRT), intensity-modulated radiation therapy (IMRT), volumetric modulated arc therapy (VMAT), image guide radiation therapy (IGRT) and other radiation therapies, and the therapeutic accuracy of these radiation therapy techniques is getting higher and higher. By applying the above radiation therapy techniques, not only can give a high dose to a tumor to improve the local control rate of the tumor, but also can reduce the dose of irradiation of normal tissue surrounding the tumor to reduce the damage to the normal tissue.

In some embodiments, a same object to be treated with radiation therapy may correspond to different radiation therapy modes. For example, for large tumors, a radiation therapy mode will be used for treatment, such as conventionally radiotherapy based on accelerator, or a high-fractionated and low-dose mode; and for small tumors, another radiation therapy mode will be used for treatment, such as precision radiotherapy based on Gamma Knife, or a low-fractionated and high-dose mode. It should be understood that due to the respective characteristics of the accelerator and the Gamma Knife, the accelerator usually uses the high-fractionated and low-dose mode for treatment, while the Gamma Knife usually uses the low-fractionated and high-dose mode for treatment. For these patients with both large and small tumors, it is necessary to use a Gamma Knife to irradiate the small tumor first, and then use an accelerator to irradiate the large tumor, so as to achieve a good radiation therapy effect.

In order to ensure that normal tissue is not irradiated by excessive doses while delivering high doses to tumors, a corresponding radiation therapy plan is usually developed for an object to be treated with radiation therapy based on a prescription dose of the object to be treated with radiation therapy. Therefore, in a case where the object to be treated with radiation therapy corresponds to different radiation therapy modes, how to generate a radiation therapy plan (i.e., a treatment plan) for the object to be treated with radiation therapy is an issue worthy of attention. Here, the radiation therapy plan is a file (document) used to control the delivery of rays by a radiation therapy device.

In light of this, embodiments of the present disclosure provide a method for generating a radiation therapy plan, by performing dose conversion on a first radiation therapy plan for an object to be treated with radiation therapy in a first radiation therapy mode, to acquire a first effective dose field obtained by the dose conversion of the first radiation therapy plan. Since an effective dose field is capable of evaluating the effect of radiation therapy on different radiation volumes in the object to be treated with radiation therapy, using the first effective dose field as the basic dose field may be more conducive to generating a second radiation therapy plan for the object to be treated with radiation therapy in a second radiation therapy mode. In this way, in a case where a same object to be treated with radiation therapy corresponds to different radiation therapy modes, according to an existing radiation therapy plan for the object to be treated with radiation therapy in one radiation therapy mode, it can be developed a radiation therapy plan for the object to be treated with radiation therapy in another radiation therapy mode. In addition, a new radiation therapy plan is developed using the effective dose field obtained by dose conversion of the existing radiation therapy plan, which may effectively improve the accuracy of the generated radiation therapy plan.

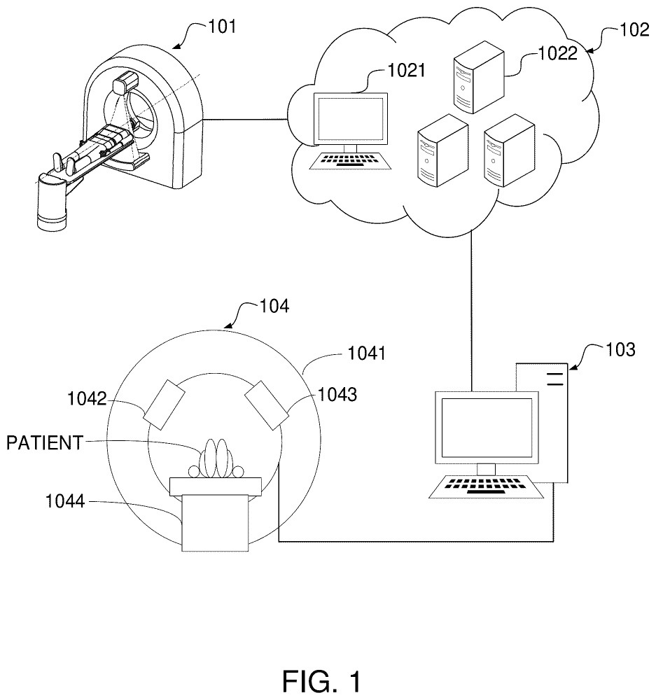

FIG. 1 is a schematic diagram showing an implementation environment of a method for generating a radiation therapy plan, in accordance with embodiments of the present disclosure. As shown in FIG. 1, the implementation environment includes an image acquisition device 101, a radiation therapy planning device 102, a control computer device 103 and a radiation therapy device 104.

Among them, the image acquisition device 101 is a device used to acquire images of the tumor site and surrounding normal tissue of an object to be treated with radiation therapy (e.g., a patient). In some embodiments, the image acquisition device 101 may be at least one of a computed tomography (CT) device, an emission computed tomography (ECT) device, a magnetic resonance imaging (MRI) device, a positron emission tomography (PET) device or an ultrasonic inspection device.

In the embodiments of the present disclosure, the image acquisition device 101 is used to acquire medical images of an object to be treated with radiation therapy, and upload the medical images of the object to the radiation therapy planning device 102, so that the radiation therapy planning device 102 performs subsequent processes of generating a radiation therapy plan based on the medical images of the object.

The radiation therapy planning device 102 is a device used to formulate, optimize and evaluate radiation therapy plans. Here, the radiation therapy planning device 102 can run a radiation therapy planning system (TPS) therein, which provides functions of developing, optimizing and evaluating radiation therapy plans. For example, the radiation therapy planning device 102 can run an RT pro TPS system therein. In addition, the TPS further provides an effective dose conversion function. For example, the effective dose conversion function may be an effective biological dose (Effective Dose in 2-Gy fractions, EQD2) conversion function or a biologically effective dose (BED) conversion function.

In some embodiments, the radiation therapy planning device 102 may include a TPS client-side 1021 and one or more TPS servers 1022.

Among them, the TPS client-side 1021 may be at least one of a smart phone, a smart watch, a desktop computer, a laptop computer, a virtual reality terminal, an augmented reality terminal, a wireless terminal, a laptop computer, or other devices. For example, in some embodiments, the one or more TPS servers 1022 run the radiation therapy planning system, and a user can trigger a dose conversion to the radiation therapy plan through the TPS client-side 1021; and then, the effective dose field obtained by the dose conversion is displayed on the TPS client-side 1021. In this way, the user's time may be effectively saved, and the converted effective dose field may be presented intuitively to facilitate the user's evaluation of the radiation therapy plan.

Here, any TPS server 1022 may be an independent physical server, or a server cluster composed of multiple physical servers or a distributed file system, or a cloud server providing at least one of basic cloud computing services such as a cloud service, a cloud database, a cloud computing, a cloud function, a cloud storage, a network service, a cloud communication, a middleware service, a domain name service, a security service, a content distribution network, and a big data or artificial intelligence platform, which are not limited in the embodiments of the present disclosure. In some embodiments, the number of the above-mentioned TPS servers 1022 may be more or less, which is not limited by the embodiments of the present disclosure. Of course, the TPS server 1022 may further include other functions to provide more comprehensive and diverse services. In some embodiments, the TPS server 1022 is used to provide background services for the above-mentioned TPS client-side 1021.

In the embodiments of the present disclosure, the radiation therapy planning device 102 is used to: acquire a first radiation therapy plan for an object that is to be treated with radiation therapy in a first radiation therapy mode; in response to a dose conversion operation on the first radiation therapy plan, acquire a first effective dose field obtained by dose conversion of the first radiation therapy plan; and generate a second radiation therapy plan for the object in a second radiation therapy mode using the first effective dose field as a basic dose field.

The control computer device 103 is a device used to control the radiation therapy device 104 to perform the radiation therapy plan. In some embodiments, the control computer device 103 may include an upper computer and a lower computer, where the upper computer is used to interact with the user, and the lower computer is used to control the movement of each moving component in the radiation therapy device 104. The upper computer may be at least one of a smart phone, a smart watch, a portable computer, a laptop, a virtual reality terminal, an augmented reality terminal, a wireless terminal, a laptop computer or a server device. The lower computer may be a control device such as a programmable logic controller (PLC).

The radiation therapy device 104 is a device used to perform radiation therapy on an object to be treated (e.g., a patient). In some embodiments, the radiation therapy device 104 may include a gantry 1041, a first treatment head 1042, a second treatment head 1043, and a support equipment 1044.

Among them, the gantry 1041 may be a rotatable gantry. The first treatment head 1042 and the second treatment head 1043 may be arranged on the gantry and used to emit treatment rays, such as gamma rays and megavolt (MV) level X-rays. For example, the first treatment head 1042 and the second treatment head 1043 may be any two of Gamma Knife treatment heads used for rotating focus radiation therapy, accelerator treatment heads used for intensity modulation conformal radiotherapy, or other radiation therapy treatment heads. In the embodiments of the present disclosure, the first treatment head 1042 and the second treatment head 1043 may correspond to different radiation therapy modes, which may be rotating focus radiation therapy and intensity modulation conformal radiotherapy, respectively. The support equipment 1014 is used to support and move the object to be treated with radiation therapy, which may be a treatment bed/couch.

In some embodiments, when the object to be treated with radiation therapy is on the support equipment 1044, the gantry 1041 rotates to drive the first treatment head 1042 and the second treatment head 1043 to perform 360-degree irradiation around the object, thereby completing the radiation therapy.

The method provided by the embodiments of the present disclosure will be introduced below based on the radiation therapy system composed of the implementation environment shown in FIG. 1.

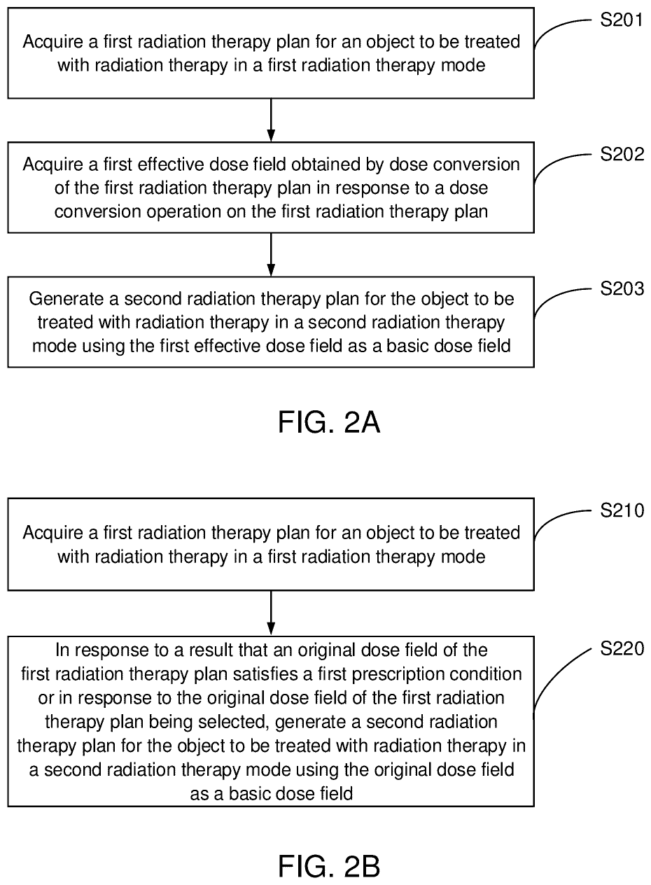

FIG. 2A is a schematic flowchart showing a method for generating a radiation therapy plan, in accordance with embodiments of the present disclosure. In some embodiments, the method for generating a radiation therapy plan is performed by an electronic device. For example, the electronic device may be the radiation therapy planning device 102 shown in FIG. 1. As shown in FIG. 2A, the method includes the following steps.

In S201, a first radiation therapy plan for an object to be treated with radiation therapy in a first radiation therapy mode is acquired.

Here, the object to be treated with radiation therapy refers to a user who is to undergo radiation therapy, such as a patient or a phantom used to simulate the patient. In the embodiments of the present disclosure, the object may correspond to different radiation therapy modes. The technical solution will be described later by taking the object to correspond to the first radiation therapy mode and the second radiation therapy mode as an example.

For example, the first radiation therapy mode and the second radiation therapy mode may be determined based on different types of treatment heads of the radiation therapy device, different types of radiation therapy modes, or radiation therapy plans with different fractions. For example, the first radiation therapy mode may be a rotating focus radiation therapy mode (or a Gamma Knife mode), and the second radiation therapy mode may be an intensity modulation conformal radiotherapy mode (or an accelerator mode). As another example, the first radiation therapy mode may be a low-fractionated and high-dose mode, and the second radiation therapy mode may be a high-fractionated and low-dose mode. The embodiments of the present disclosure do not limit the specific forms of the first radiation therapy mode and the second radiation therapy mode.

In S202, in response to a dose conversion operation on the first radiation therapy plan, a first effective dose field obtained by dose conversion of the first radiation therapy plan is acquired.

In the embodiments of the present disclosure, an effective dose field is used to evaluate the effect of radiation therapy on different radiation volumes in the object to be treated with radiation therapy. Accordingly, the first effective dose field is used to evaluate the effect of radiation therapy based on the first radiation therapy plan on different radiation volumes in the object. Here, the radiation area refers to biological tissue, such as organs.

The effective dose field refers to an effective biological dose field, such as an EQD2 dose field or a BED dose field. It should be understood that the dose conversion refers to a conversion of different radiation doses into an effective biological dose. In this way, it is possible to compare the effects of different types of radiation therapy schemes more accurately on biological tissue. Here, taking into account the extent of cell damage after an organism is exposed to radiation, the effective biological dose refers to a dose obtained by converting different radiation doses. For example, the larger the converted dose, the more severe the damage to biological cells.

It should be understood that a dose field refers to the distribution of radiation dose used in radiation therapy, which is used to characterize the distribution situation of radiation energy released by radioactive rays or other radiation sources in the human body. For example, the definition of the dose field can include aspects such as the intensity, position, and orientation of the dose distribution. Typically, the dose field can be generated according to the radiation therapy plan and can be displayed as a computer image or printed out to help medical personnel (such as radiotherapists) accurately determine the delivery volume and shape of the radiation dose, thereby ensuring the accuracy and safety of radiation therapy.

In S203, using the first effective dose field as a basic dose field, a second radiation therapy plan for the object to be treated with radiation therapy in a second radiation therapy mode is generated.

In the technical solution provided by the embodiments of the present disclosure, by performing the dose conversion on the first radiation therapy plan for the object to be treated with radiation therapy in the first radiation therapy mode, the first effective dose field obtained by the dose conversion of the first radiation therapy plan is acquired. Since the effective dose field is capable of evaluating the effect of radiation therapy on different radiation volumes in the object to be treated with radiation therapy, using the first effective dose field as the basic dose field may be conducive to generating the second radiation therapy plan for the object to be treated with radiation therapy in the second radiation therapy mode. In this way, in the case where the same object to be treated with radiation therapy corresponds to different radiation therapy modes, according to an existing radiation therapy plan for the object to be treated with radiation therapy in one radiation therapy mode, it can be developed a radiation therapy plan for the object to be treated with radiation therapy in another radiation therapy mode. In addition, a new radiation therapy plan is developed using the effective dose field obtained by dose conversion of the existing radiation therapy plan, which may effectively improve the accuracy of the generated radiation therapy plan.

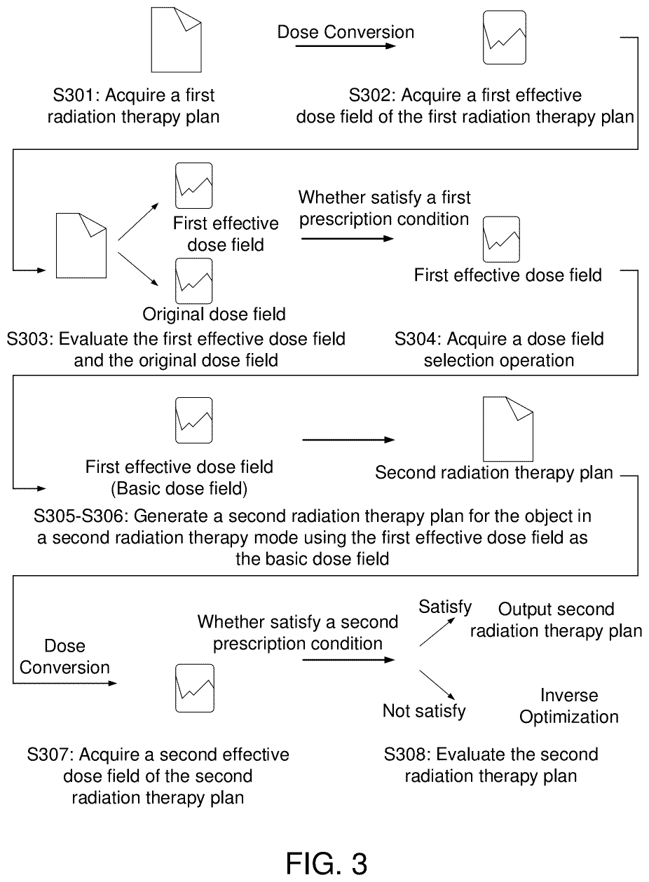

FIG. 2A above-mentioned shows a briefness implementation of the present disclosure. The method for generating a radiation therapy plan provided by the present disclosure will be described in detail below based on a specific implementation. FIG. 3 is a schematic flowchart showing a method for generating a radiation therapy plan, in accordance with some embodiments of the present disclosure. In some embodiments, the method for generating a radiation therapy plan is performed by an electronic device. For example, the electronic device may be the radiation therapy planning device 102 shown in FIG. 1. As shown in FIG. 3, with the radiation therapy planning device as an execution object, the method includes the following steps.

In S301, the radiation therapy planning device acquires a first radiation therapy plan for an object to be treated with radiation therapy in a first radiation therapy mode.

Here, the object to be treated with radiation therapy refers to a user who is to undergo radiation therapy, such as a patient or a phantom used to simulate the patient. In the embodiments of the present disclosure, the object may correspond to different radiation therapy modes. The technical solution will be described later by taking the object to correspond to the first radiation therapy mode and the second radiation therapy mode as an example.

For example, the first radiation therapy mode and the second radiation therapy mode may be determined based on different types of treatment heads of the radiation therapy device, different types of radiation therapy modes, or radiation therapy plans with different fractions. For example, the first radiation therapy mode may be a Gamma Knife mode, and the second radiation therapy mode may be an accelerator mode. As another example, the first radiation therapy mode may be a low-fractionated and high-dose mode, and the second radiation therapy mode may be a high-fractionated and low-dose mode.

The embodiments of the present disclosure do not limit the specific forms of the first radiation therapy mode and the second radiation therapy mode.

In some embodiments, a process for generating the first radiation therapy plan may refer to the following actions (1-1) to (1-3).

In action (1-1), the radiation therapy planning device obtains medical image (or images) of the object to be treated with radiation therapy.

Here, the medical image is used to present the tumor site and surrounding normal tissue of the object in the form of images. For example, the medical image (or images) may include at least one of a CT scan image, an MRI scan image, or an ultrasound image.

In some embodiments, a process of obtaining the medical image of the object by the radiation therapy planning device may be referred to any of the following two possible implementations.

In a possible implementation, after acquiring the medical image of the object, the image acquisition device transmits the medical image of the object to the radiation therapy planning device. Accordingly, the radiation therapy planning device receives the medical image from the image acquisition device, that is, the radiation therapy planning device obtains the medical image of the object.

In another possible implementation, after acquiring the medical image of the object, the image acquisition device outputs the medical image of the object. For example, the medical image output may be in the form of a data package file or a physical image picture in this case. Furthermore, the medical personnel upload the medical image output by the image acquisition device to the radiation therapy planning device, so the radiation therapy planning device obtains the medical image of the object.

In the above embodiments, two implementations for the radiation therapy planning device to obtain the medical image of the object to be treated with radiation therapy are provided, which improves the flexibility of obtaining the medical image. It is worth noting that in some other embodiments, the radiation therapy planning device may also adopt other implementations to obtain the medical image of the object. The embodiments of the present disclosure do not limit the process for obtaining the medical image.

In action (1-2), the radiation therapy planning device contours a target volume based on the medical image of the object to be treated with radiation therapy to obtain a contoured image.

Here, the contoured image is used to characterize an outline of the target volume or an organ.

In some embodiments, after obtaining the medical image of the object to be treated with radiation therapy, the radiation therapy planning device automatically contours the target volume based on the medical image of the object to be treated with radiation therapy, so as to obtain the contoured image.

Alternatively, in some other embodiments, after obtaining the medical image of the object to be treated with radiation therapy, the radiation therapy planning device displays the medical image of the object to be treated with radiation therapy. And then the medical personnel perform target volume contouring based on the medical image of the object to be treated with radiation therapy, so as to obtain the contoured image.

In the above embodiments, two methods are provided, in which the radiation therapy planning device automatically contours the target volume and the medical personnel manually delineate the target volume, which improves the flexibility of obtaining the contoured image. It is worth noting that in some other embodiments, the radiation therapy planning device may also adopt other implementations to perform the target volume contouring to obtain the contoured image. The embodiments of the present disclosure do not limit the process for obtaining the contoured image.

In action (1-3), the radiation therapy planning device creates a first radiation therapy plan based on the contoured image, determines a first objective function based on the first prescription dose of the first radiation therapy mode, and then performs inverse optimization on the first objective function to obtain the first radiation therapy plan.

It will be noted that the “creates” in “creates a first radiation therapy plan” in (1-3) means that the user clicks a “NEW” button through a TPS software interface, for example, which is an operation that initiates the generation of the first radiation therapy plan for the purpose of generating the first radiation therapy plan through subsequent steps, rather than actually generating the first radiation therapy plan.

In some embodiments, after creating the first radiation therapy plan based on the contoured image, the radiation therapy planning device can determine a dose-volume histogram (DVH) corresponding to the first radiation therapy mode based on the first prescription dose of the first radiation therapy mode. Furthermore, the inverse optimization is performed on the dose-volume histogram serving as the first objective function, to obtain the first radiation therapy plan.

Here, the dose-volume histogram is used to characterize how much volume within the volume of the target volume and organ is irradiated at what high dose level.

Alternatively, in some other embodiments, after creating the first radiation therapy plan based on the contoured image, the radiation therapy planning device can determine dose distribution corresponding to the first radiation therapy mode based on the first prescription dose of the first radiation therapy mode. Furthermore, the inverse optimization is performed on the dose distribution serving as the first objective function, to obtain the first radiation therapy plan.

Here, the dose distribution is used to characterize the dose distribution in different sites of the target volume of the object to be treated with radiation therapy. For example, the dose distribution may be in the form of an image/map, a table, or a curve. For example, the dose distribution may be a three-dimensional dose distribution map taking the form of an image as an example.

Alternatively, in still some other embodiments, after creating the first radiation therapy plan based on the contoured image, the radiation therapy planning device can determine a fluence map corresponding to the first radiation therapy mode based on the first prescription dose of the first radiation therapy mode. Furthermore, the inverse optimization is performed on the fluence map serving as the first objective function, to obtain the first radiation therapy plan.

Here, the fluence map is used to characterize the intensity distribution of radioactive rays emitted by the radiation therapy device at different position points (x, y) in the target volume of the object to be treated with radiation therapy.

The process of generating the first radiation therapy plan is described above by considering an example in which the inverse optimization is performed on one of the dose-volume histogram, the dose distribution and the fluence map which serves as the first objective function. It will be noted that the radiation therapy planning device may also perform a process of the inverse optimization based on two or three items of the dose-volume histogram, the dose distribution and the fluence map (that is, the first objective function may include two or three items of the foregoing) to generate the first radiation therapy plan. The embodiments of the present disclosure do not limit this. It is worth noting that in some other embodiments, the radiation therapy planning device may also adopt other implementations to perform the process of the inverse optimization to generate the first radiation therapy plan. The embodiments of the present disclosure do not limit the specific process of the inverse optimization.

It will be noted that the first radiation therapy plan may be generated in real time in a case of implementing the technical solution. For example, in a case of implementing the technical solution, the above (1-1) to (1-3) are used to acquire the first radiation therapy plan of the object to be treated with radiation therapy in the first radiation therapy mode. Alternatively, the first radiation therapy plan may also be generated in advance before implementing the technical solution. For example, a radiation therapy plan record may be maintained in the radiation therapy planning device, which is used to store respective existing radiation therapy plans for multiple objects to be treated with radiation therapy. In a case of implementing the technical solution, based on an identification of a certain object to be treated with radiation therapy, the radiation therapy planning device can acquire a radiation therapy plan corresponding to the identification of this object from the radiation therapy plan record as the first radiation therapy plan. The identification of the object to be treated with radiation therapy may be at least one of a name, a serial number, or an identification number (ID).

In S302, in response to a dose conversion operation on the first radiation therapy plan, the radiation therapy planning device acquires a first effective dose field obtained by dose conversion of the first radiation therapy plan.

In the embodiments of the present disclosure, an effective dose field is used to evaluate the effect of radiation therapy on different radiation volumes in the object to be treated with radiation therapy. Accordingly, the first effective dose field is used to evaluate the effect of radiation therapy based on the first radiation therapy plan on different radiation volumes in the object. Here, the radiation area refers to biological tissue, such as organs.

The effective dose field refers to an effective biological dose field, such as an EQD2 dose field, or a BED dose field. It should be understood that the dose conversion refers to a conversion of different radiation doses into an effective biological dose. In this way, it is possible to compare the effects of different types of radiation therapy schemes more accurately on biological tissue. Here, taking into account the extent of cell damage after an organism is exposed to radiation, the effective biological dose refers to a dose obtained by converting different radiation doses. For example, the larger the converted dose, the more severe the damage to biological cells.

It should be understood that a dose field refers to the distribution of radiation dose used in radiation therapy, which is used to characterize the distribution situation of radiation energy released by radioactive rays or other radiation sources in the human body. For example, the definition of the dose field can include aspects such as the intensity, position, and orientation of the dose distribution. Typically, the dose field can be generated according to the radiation therapy plan and can be displayed as a computer image or printed out to help medical personnel (such as radiotherapists) accurately determine the delivery volume and shape of the radiation dose, thereby ensuring the accuracy and safety of radiation therapy.

In some embodiments, a display interface of the radiation therapy planning device may be provided thereon with a functional control for dose conversion. By performing a triggering operation on the functional control for dose conversion, the medical personnel can trigger the dose conversion operation of the first radiation therapy plan, so that the radiation therapy planning device performs the process of the dose conversion subsequently. The triggering operation may be any of a single-click operation, a double-click operation, a long press operation or a floating operation.

In some embodiments, in response to the dose conversion operation on the first radiation therapy plan, the radiation therapy planning device determines an effective dose corresponding to each radiation volume corresponding to the first radiation therapy plan, based on a radiation dose of each radiation volume, a fractionated radiation dose of each radiation volume, and a radiosensitivity parameter corresponding to each radiation volume, to obtain the first effective dose field.

Among them, the radiation dose of each radiation volume refers to the total radiation dose of the radiation volume, such as the total radiation dose of each organ. In some embodiments, the radiation dose of each radiation volume may be determined based on an original dose field of the first radiation therapy plan. The fractionated radiation dose of each radiation volume may be set by medical personnel before implementing the technical solution. The radiosensitivity parameter corresponding to each radiation volume may be determined based on a preset sensitivity constant table, such as an organ sensitivity constant table. Here, the sensitivity constant table is used to record radiosensitivity parameters corresponding to identifications of different radiation volumes (such as organs). For example, based on an identification of each radiation volume corresponding to the first radiation therapy plan, the radiosensitivity parameter corresponding to the identification of each radiation volume may be determined from the sensitivity constant table, so as to obtain the radiosensitivity parameter corresponding to each radiation volume.

For example, taking the first effective dose field as the EQD2 dose field as an example, the process of the dose conversion may be: determining an effective dose corresponding to each radiation volume corresponding to the first radiation therapy plan, based on a radiation dose of each radiation volume, a fractionated radiation dose of each radiation volume, a radiosensitivity parameter corresponding to each radiation volume, and the following formula (1), to obtain the first effective dose field.

$$EQD 2 = D * ( d + α / β ) / ( 2 + α / β ) \qquad Formula ( 1 )$$

In formula (1), EQD2 represents the effective dose corresponding to the radiation volume; D represents the radiation dose of the radiation volume; d represents the fractionated radiation dose of the radiation volume; and α/β represents the radiosensitivity parameter corresponding to the radiation volume. It should be understood that for different radiation volumes, the α/β are different; and 2 represents the dose conversion of 2Gy (Gray) per fraction, which may also be set to other values.

In this way, based on the above formula (1), the effective dose corresponding to each radiation volume can be determined, so that the first effective dose field can be obtained. In this case, the radiation therapy plan corresponding to the first effective dose field may be called an EQD2 plan.

Further, in some embodiments, after acquiring the first effective dose field obtained by dose conversion of the first radiation therapy plan, the radiation therapy planning device may display the first effective dose field, so that medical personnel can intuitively view the converted first effective dose field subsequently.

In S303, the radiation therapy planning device evaluates whether an original dose field of the first radiation therapy plan and the first effective dose field satisfy a first prescription condition.

Here, the first prescription condition is used to evaluate the respective quality of the original dose field and the first effective dose field. In some embodiments, the first prescription condition may be set such that a target coverage is greater than a preset coverage, in which the target coverage refers to a rate of the volume of a target volume that reaches the prescription dose to the total volume of the target volume. The preset coverage may be a target coverage required by the prescription. For example, the preset coverage may be 95%, which means that the rate of the volume of the target volume that reaches the prescription dose to the total volume of the target volume should reach 95%. It should be understood that if the target coverage rate of the radiation therapy plan is greater than the preset coverage, it means that the radiation therapy plan satisfies the first prescription condition; and if the target coverage rate of the radiation therapy plan is less than or equal to the preset coverage, it means that the radiation therapy plan does not satisfy the first prescription condition.

In some embodiments, the radiation therapy planning device determines a target coverage corresponding to the original dose field of the first radiation therapy plan based on the original dose field, and determines a target coverage corresponding to the first effective dose field based on the first effective dose field. Further, whether each of the original dose field and the first effective dose field satisfies the first prescription condition is evaluated by determining whether the respective target coverage of the first effective dose field and the original dose field is greater than the preset coverage.

Further, in some embodiments, the radiation therapy planning device can display the evaluation result of the evaluation, so that the medical personnel can intuitively view the evaluation result of the evaluating subsequently, thereby performing a dose field selection operation based on the evaluation result.

In S304, the radiation therapy planning device acquires the dose field selection operation based on the evaluation result of the evaluating.

Here, the dose field selection operation refers to an operation of selecting a certain dose field. For example, the dose field selection operation may be an operation indicating that the first effective dose field is selected; alternatively, the dose field selection operation may be an operation indicating that the original dose field of the first radiation therapy plan is selected.

In some embodiments, the radiation therapy planning device acquires an operation for indicating that the first effective dose field is selected in a case where the evaluation result indicates that the first effective dose field satisfies the first prescription condition. Furthermore, a process of generating a second radiation therapy plan is performed based on the selected first effective dose field subsequently.

Alternatively, in some other embodiments, the radiation therapy planning device acquires an operation for indicating that the original dose field of the first radiation therapy plan is selected in a case where the evaluation result indicates that the original dose field of the first radiation therapy plan satisfies the first prescription condition. Furthermore, a process of generating a second radiation therapy plan is performed based on the selected original dose field of the first radiation therapy plan subsequently.

For example, in another method for generating a radiation therapy plan provided by embodiments of the present disclosure, as shown in FIG. 2B, the method includes the following steps: S210, acquiring a first radiation therapy plan for an object to be treated with radiation therapy in a first radiation therapy mode; and S220, in response to a result that an original dose field of the first radiation therapy plan satisfies a first prescription condition, generating a second radiation therapy plan for the object to be treated with radiation therapy in a second radiation therapy mode using the original dose field as a basic dose field. Here, the process of generating the first radiation therapy plan may refer to the aforementioned actions (1-1) to (1-3), which will not be described again here.

In S305, among an original dose field of the first radiation therapy plan and the first effective dose field, the radiation therapy planning device uses the first effective dose field as the basic dose field if determining that the first effective dose field is selected.

Here, the basic dose field can also be called a background dose field. For example, taking a basic dose field as 20-20-20 as an example, if a dose required by the prescription is 100-100-100, the plan needs to be optimized based on the basic dose field of 20-20-20 to make the dose reach 100-100-100. Of course, if the basic dose field is not provided, the plan needs to be optimized on the basis of 0-0-0 to make the dose reach 100-100-100. It should be understood that the dose field is a multi-dimensional matrix. Taking the basic dose field 20-20-20 as an example, each value “20” is a dose in one dimension, and its unit may be cGy or Gy, for example.

The above S305 takes the case where the first effective dose field is selected as an example.

Alternatively, in some other embodiments, among the original dose field of the first radiation therapy plan and the first effective dose field, the radiation therapy planning device uses the original dose field as the basic dose field if determining that the original dose field is selected.

For example, in a method for generating another radiation therapy plan provided by embodiments of the present disclosure, as shown in FIG. 2B, the method includes the following steps: S210, acquiring a first radiation therapy plan for an object to be treated with radiation therapy in a first radiation therapy mode; and S220, in response to the original dose field of the first radiation therapy plan being selected, generating a second radiation therapy plan for the object to be treated with radiation therapy in a second radiation therapy mode using the original dose field as a basic dose field. Here, the process of generating the first radiation therapy plan may refer to the aforementioned actions (1-1) to (1-3), which will not be described again here.

The embodiments shown by S304 and S305 above take the process of determining the basic dose field based on the dose field selection operation as an example to illustrate the technical solution. After acquiring the evaluation result of the evaluation based on S303, the medical personnel can perform analysis based on the evaluation result of the evaluation, and implement the dose field selection operation on the interface of the radiation therapy planning device to determine the basic dose field as selected.

In some other embodiments, after acquiring the evaluation result based on S303, the radiation therapy planning device may also automatically select the basic dose field. The corresponding process may be that: in response to the evaluation result indicating that the first effective dose field satisfies the first prescription condition, the radiation therapy planning device automatically uses the first effective dose field as the basic dose field; alternatively, in response to the evaluation result indicating that the original dose field of the first radiation therapy plan satisfies the first prescription condition, the radiation therapy planning device automatically uses the original dose field as the basic dose field (as shown in S220). In this way, an automatic selection of the basic dose field is realized and the efficiency of human-computer interaction (HCl) is improved.

In S306, the radiation therapy planning device generates a second radiation therapy plan for the object to be treated with radiation therapy in a second radiation therapy mode.

In embodiments shown by S305 and S306 above, the radiation therapy planning device uses the first effective dose field as the basic dose field to generate the second radiation therapy plan for the object to be treated with radiation therapy in the second radiation therapy mode.

In some embodiments, a process of generating the second radiation therapy plan may be: creating a second radiation therapy plan, determining a second objective function based on a second prescription dose of the second radiation therapy mode, and then performing inverse optimization on the second objective function using the first effective dose field as the basic dose field, so as to obtain the second radiation therapy plan.

It will be noted that, similarly, the “creates” in “creates a second radiation therapy plan” in the above process means that the user clicks the “NEW” button through the TPS software interface, for example, which is an operation that initiates the generation of the second radiation therapy plan for the purpose of generating the second radiation therapy plan through subsequent steps, rather than actually generating the second radiation therapy plan.

In a possible implementation, after creating the second radiation therapy plan, the radiation therapy planning device can determine a dose-volume histogram (DVH) corresponding to the second radiation therapy mode based on the second prescription dose of the second radiation therapy mode. Furthermore, the inverse optimization is performed on the dose-volume histogram serving as the second objective function, to obtain the second radiation therapy plan.

Alternatively, in another possible implementation, after creating the second radiation therapy plan, the radiation therapy planning device can determine dose distribution corresponding to the second radiation therapy mode based on the second prescription dose of the second radiation therapy mode. Furthermore, the inverse optimization is performed on the dose distribution serving as the second objective function, to obtain the second radiation therapy plan.

Alternatively, in yet another possible implementation, after creating the second radiation therapy plan, the radiation therapy planning device can determine a fluence map corresponding to the second radiation therapy mode based on the second prescription dose of the second radiation therapy mode. Furthermore, the inverse optimization is performed on the fluence map serving as the second objective function, to obtain the second radiation therapy plan.

The process of generating the second radiation therapy plan is described above by considering an example in which the inverse optimization is performed on one of the dose-volume histogram, the dose distribution and the fluence map which serves as the second objective function. It will be noted that the radiation therapy planning device may also perform a process of the inverse optimization based on two or three items of the dose-volume histogram, the dose distribution and the fluence map (that is, the second objective function may include two or three items of the foregoing) to generate the second radiation therapy plan. The embodiments of the present disclosure do not limit this. It is worth noting that in some other embodiments, the radiation therapy planning device may also adopt other implementations to perform the process of the inverse optimization to generate the second radiation therapy plan. The embodiments of the present disclosure do not limit the specific process of the inverse optimization.

In addition, it will be noted that S303 to S305 above may be optional steps. In some embodiments, after acquiring the first effective dose field based on S302, the radiation therapy planning device does not need to perform the steps of S303 to S305 described above, and automatically uses the first effective dose field as the basic dose field. In these embodiments, the first effective dose field may not satisfy the first prescription condition. In this case, the inverse optimization may be performed on the first objective function again until the first effective dose field satisfies the first prescription condition, then the subsequent process of generating the second radiation therapy plan is performed based on the first radiation therapy plan that satisfies the first prescription condition.

Alternatively, in a case where the first effective dose field does not satisfy the first prescription condition, the subsequent process of generating the second radiation therapy plan may also be performed. In this case, the first radiation therapy plan can be supplemented during the optimization process of the second radiation therapy plan. The corresponding process may be that: the radiation therapy planning device determines a dose difference value of a target radiation volume in radiation volumes corresponding to the first radiation therapy plan, and increases the effective dose of the target radiation volume corresponding to the second radiation therapy plan by a dose corresponding to the dose difference value, based on the dose difference value of the target radiation volume. Here, the target radiation volume refers to a radiation volume not reached a first prescription dose of the first radiation therapy mode; and the dose difference value represents a difference value between an effective dose of the target radiation volume and the first prescription dose. In this way, the accuracy of generating radiation therapy plans may be ensured.

In S307, in response to the dose conversion operation on the second radiation therapy plan, the radiation therapy planning device acquires a second effective dose field obtained by dose conversion of the second radiation therapy plan.

In some embodiments, a display interface of the radiation therapy planning device may be provided thereon with a functional control for dose conversion. By performing a triggering operation on the functional control for dose conversion, medical personnel can trigger the dose conversion operation of the second radiation therapy plan, so that the radiation therapy planning device performs the process of the dose conversion subsequently.

In some embodiments, in response to the dose conversion operation on the second radiation therapy plan, the radiation therapy planning device determines an effective dose corresponding to each radiation volume corresponding to the second radiation therapy plan, based on a radiation dose of each radiation volume, a fractionated radiation dose of each radiation volume, and a radiosensitivity parameter corresponding to each radiation volume, to obtain the second effective dose field.

Among them, the radiation dose of each radiation volume refers to the total radiation dose of the radiation volume. In some embodiments, the radiation dose of each radiation volume may be determined based on an original dose field of the second radiation therapy plan. The fractionated radiation dose of each radiation volume may be set by medical personnel before implementing this technical solution. The radiosensitivity parameter corresponding to each radiation volume may be determined based on a preset sensitivity constant table, such as an organ sensitivity constant table.

Further, in some embodiments, after acquiring the second effective dose field obtained by dose conversion of the second radiation therapy plan, the radiation therapy planning device may display the second effective dose field, so that medical personnel can intuitively view the converted second effective dose field subsequently.

In S308, the radiation therapy planning device evaluates whether the second radiation therapy plan satisfies a second prescription condition, based on the second effective dose field.

Here, the second prescription condition is used to evaluate the quality of the second radiation therapy plan. For example, the second prescription condition may be the same as the first prescription condition, and both are set such that the target coverage is greater than the preset coverage. Alternatively, the second prescription condition may be set to other conditions, such as at least one of that the target isocenter is the same as the device isocenter, the target arc range is the same as the device arc range (or the device arc range includes the target arc range), the device rotate speed (i.e., machine rotate speed) satisfies the target irradiation duration, or the device rotate speed satisfies the device arc irradiation duration. The embodiments of the present disclosure do not limit the setting of the second prescription condition.

In some embodiments, in a case where the second radiation therapy mode of the object to be treated with radiation therapy is a preset radiation therapy mode, S307 and S308 above are executed to evaluate the second radiation therapy mode. For example, taking the preset radiation therapy mode as a high-dose and low-fractionated dose mode of the accelerator, in a case where the second radiation therapy mode of the object to be treated with radiation therapy is the high-dose and low-fractionated dose mode of the accelerator, S307 and S308 are executed to evaluate the second radiation therapy mode.

Furthermore, if the second radiation therapy plan satisfies the second prescription condition, the second radiation therapy plan is output. If the second radiation therapy plan does not satisfy the second prescription condition, the inverse optimization may be performed on the second objective function again until the second radiation therapy plan satisfies the second prescription condition, and then the second radiation therapy plan is output.

It will be noted that S307 and S308 above may be optional steps. In some embodiments, after the second radiation therapy plan is generated based on S306, there is no need to perform S307 and S308, and the second radiation therapy plan is output directly.

In some embodiments, after acquiring the second effective dose field obtained by dose conversion of the second radiation therapy plan, the first radiation therapy plan and the second radiation therapy plan may further be comprehensively evaluated. The corresponding process may be: performing superposition processing on the first effective dose field and the second effective dose field to obtain a comprehensive effective dose field of the first radiation therapy plan and the second radiation therapy plan; and evaluating whether comprehensive treatment effect of the first radiation therapy plan and the second radiation therapy plan satisfies a third prescription condition based on the comprehensive effective dose field.

Here, the setting of the third prescription condition is similar to that of the second prescription condition. In some examples, at least any two of the first prescription condition, the second prescription condition and the third prescription condition may be the same, that is, the first prescription condition is the same as the second prescription condition, but different from the third prescription condition; alternatively, the first prescription condition is the same as the third prescription condition, but different from the second prescription condition; alternatively, the second prescription condition is the same as the third prescription condition, but different from the first prescription condition. For example, the third prescription condition may be the same as the first prescription condition (or the same as both the first prescription condition and the second prescription condition), all of which are set such that the target coverage is greater than the preset coverage. Alternatively, the first prescription condition, the second prescription condition, and the third prescription condition are the same. In some other examples, the first prescription condition, the second prescription condition, and the third prescription condition are different from one another. In addition, the third prescription condition may also be set to other conditions, such as at least one of that the target isocenter is the same as the device isocenter, the target arc range is the same as the device arc range (or the device arc range includes the target arc range), the device rotate speed (i.e., machine rotate speed) satisfies the target irradiation duration, or the device rotate speed satisfies the device arc irradiation duration. The embodiments of the present disclosure do not limit the specific settings of the above three prescription conditions, which can be flexibly adjusted according to the specific radiation therapy plans.

Furthermore, if the comprehensive treatment effect of the first radiation therapy plan and the second radiation therapy plan satisfies the third prescription condition, the second radiation therapy plan is output. If the comprehensive treatment effect of the first radiation therapy plan and the second radiation therapy plan does not satisfy the third prescription condition, the inverse optimization may be performed on the second objective function again until the comprehensive treatment effect of the first radiation therapy plan and the second radiation therapy plan satisfies the third prescription condition, and then the second radiation therapy plan is output.

In some embodiments of the present disclosure, by providing an effective dose conversion function in the radiation therapy planning system, and then combining the first effective dose field and the second effective dose field through dose conversion, it is possible to evaluate the dose effect of radiation therapy quickly and easily in combination with the two radiation therapy plans, thereby improving treatment efficacy. Compared with the related art where users manually calculate effective doses or use third-party tools to calculate effective doses, these embodiments of the present disclosure greatly improve the efficiency of dose conversion.

For example, the first radiation therapy mode and the second radiation therapy mode respectively correspond to different treatment heads of the radiation therapy device. For example, a treatment head corresponding to the first radiation therapy mode is a Gamma Knife, and a treatment head corresponding to the second radiation therapy mode is an accelerator. The process of developing an Accelerator-Gamma Knife hybrid radiation therapy plan (or called “Double-Head” plan) may be that: first, patient data such as medical image (images) and contour image (images) that are required for the formulation of radiation therapy plans are imported into the radiation therapy planning device, and a Gamma Knife plan is generated by combining a Gamma Knife prescription dose set by medical personnel; a dose conversion is then performed based on the Gamma Knife plan, so as to obtain an effective dose field of the Gamma Knife plan, after which an accelerator plan is generated using the effective dose field of the Gamma Knife plan as a background dose field (i.e., the basic dose field); and then, the Gamma Knife plan and the accelerator plan are superimposed to output the Double-Head plan. In this way, a process for the formulation of Double-Head plan is provided. In this regard, in a case of determining the background dose field, the Gamma Knife plan may be directly dose-converted into intuitive dose field data for easy evaluation by the medical personnel.

In addition, in some embodiments, the medical personnel can evaluate the respective radiation therapy effect of the original dose field and the effective dose field of the Gamma Knife plan, and select the one with better radiation therapy effect (for example, satisfying the prescription condition) as the background dose field, so as to formulate an accelerator program.

In the technical solution provided by the embodiments of the present disclosure, by performing the dose conversion on the first radiation therapy plan for the object to be treated with radiation therapy in the first radiation therapy mode, the first effective dose field obtained by the dose conversion of the first radiation therapy plan is acquired. Since the effective dose field is capable of evaluating the effect of radiation therapy on different radiation volumes in the object to be treated with radiation therapy, using the first effective dose field as the basic dose field may be conducive to generating the second radiation therapy plan for the object to be treated with radiation therapy in the second radiation therapy mode. In this way, in the case where the same object to be treated with radiation therapy corresponds to different radiation therapy modes, according to an existing radiation therapy plan for the object to be treated with radiation therapy in one radiation therapy mode, it can be developed a radiation therapy plan for the object to be treated with radiation therapy in another radiation therapy mode. In addition, a new radiation therapy plan is developed using the effective dose field obtained by dose conversion of the existing radiation therapy plan, which may effectively improve the accuracy of the generated radiation therapy plan.

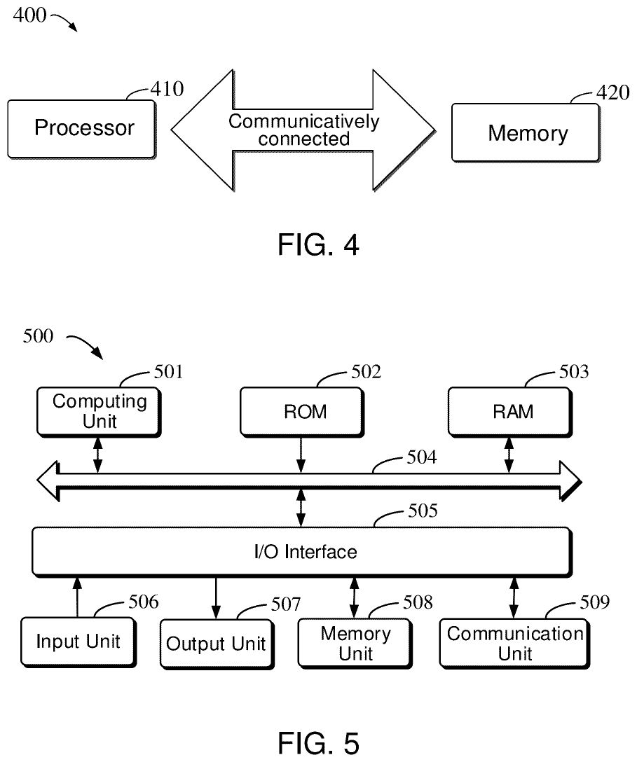

Embodiments of the present disclosure provide an electronic device. As shown in FIG. 4, the electronic device 400 includes at least one processor 410 and a memory 420 communicatively connected to the at least one processor 410. The memory 420 stores instructions executable by the at least one processor 410, when executed by the at least one processor 420, cause the at least one processor 420 to perform the method for generating a radiation therapy plan provided by any of the above embodiments of the present disclosure.

Embodiments of the present disclosure provide a non-transitory computer-readable storage medium storing computer instructions, in which the computer instructions are used to cause an electronic device to perform the method for generating a radiation therapy plan provided by any of the above embodiments of the present disclosure.

Embodiments of the present disclosure provide a computer program product, including a computer program that, when executed by a processor, implements the method for generating a radiation therapy plan provided by any of the above embodiments of the present disclosure.

In some embodiments, the electronic device may be the radiation therapy planning device shown in FIG. 1 above. FIG. 5 is a block diagram showing an exemplary electronic device 500 that may be used to implement embodiments of the present disclosure. The electronic device 500 is intended to represent digital computers of various forms, such as a laptop computer, a desktop computer, a workstation, a personal digital assistant, a server, a blade server, a mainframe computer, and other suitable computers. The electronic device 500 may also represent mobile devices of various forms, such as a personal digital assistant, a cellular phone, a smart phone, a wearable device, and other similar computing device. The components shown herein, their connections and relationships, and their functions are examples only and are not intended to limit implementations of the disclosure described and/or claimed herein.

As shown in FIG. 5, the electronic device 500 includes a computing unit 501, which may perform various appropriate actions and processes according to a computer program stored in a read-only memory (ROM) 502 or loaded from a memory unit 508 into a random access memory (RAM) 503. Various programs and data necessary for the operation of the electronic device 500 may also be stored in the RAM 503. The computing unit 501, the ROM 502 and the RAM 503 are connected to each other via a bus 504. An input/output (I/O) interface 505 is also connected to the bus 504.

There are various components connected to the I/O interface 505 in the electronic device 500, which include: an input unit 506, such as a keyboard, a mouse, and a touch pad; an output unit 507, such as various types of displays and speakers; a memory unit 508, such as a disk and an optical disk; and a communication unit 509, such as a network card, a modem, and a wireless communication transceiver. Furthermore, the communication unit 509 allows the electronic device 500 to exchange information/data with other devices through computer networks such as the Internet and/or various telecommunications networks.

The computing unit 501 may be a variety of general and/or special purpose processing components having processing and computing capabilities. Some examples of the computing unit 501 include, but are not limited to, a central processing unit (CPU), a graphics processing unit (GPU), various dedicated artificial intelligence (AI) computing chips, various computing units running machine learning model algorithm, digital signal processor (DSP), and any appropriate processor, controller, microcontroller, etc. The Computing unit 501 performs various methods and processes described above, such as the method for generating a radiation therapy plan. For example, in some embodiments, the method for generating a radiation therapy plan may be implemented as a computer software program, which is tangibly included in a machine-readable medium, such as in the memory unit 508. In some embodiments, part or all of the computer program may be loaded and/or installed onto the electronic device 500 via the ROM 502 and/or the communication unit 509. When the computer program is loaded into the RAM 503 and executed by the computing unit 501, one or more steps of the method for generating a radiation therapy plan described above may be performed. Alternatively, in some other embodiments, the computing unit 501 may be configured to perform the method for generating a radiation therapy plan in any other suitable manner (e.g., by means of firmware).

Various implementations of the systems and techniques described above may be realized in a digital electronic circuit system, an integrated circuit system, a field programmable gate array (FPGA), an application specific integrated circuit (ASIC), an application specific standard part (ASSP), a system on chip (SOC), a complex programmable logic device (CPLD), computer hardware, firmware, software, and/or their combination. These various embodiments may include implementations in one or more computer programs, the one or more computer programs are executable and/or interpreted on a programmable system including at least one programmable processor. The programmable processor, which may be a dedicated or general-purpose programmable processor, may receive data and instructions from a memory system, at least one input device, and at least one output device, and transmit data and instructions to the memory system, the at least one input device, and the at least one output device.

Program code for implementing the methods of the present disclosure may be written in any combination of one or more programming languages. Such program code may be provided to a processor or controller of a general-purpose computer, a special-purpose computer, or other programmable data processing device such that the program code, when executed by the processor or controller, causes the functions/operations set forth in the flowcharts and/or block diagrams to be performed. The program code may be executed entirely on the machine, partially on the machine, partially on the machine as a stand-alone software package and partially on a remote machine, or entirely on a remote machine or server.

In the context of the present disclosure, a machine-readable medium may be a tangible medium that may contain or store a program for use by or in connection with instructions to execute system, apparatus, or device. The machine-readable medium may be a machine-readable signal medium or a machine-readable storage medium. Machine-readable media may include, but are not limited to, electronic, magnetic, optical, electromagnetic, infrared, or semiconductor systems, apparatuses or devices, or any suitable combination of the foregoing. More specific examples of the machine-readable storage medium may include an electrical connection based on one or more wires, a portable computer disk, a hard disk, a random-access memory, a read-only memory, an erasable programmable read only memory (EPROM or flash memory), an optical fiber, a convenient compact disc read-only memory (CD-ROM), an optical storage device, a magnetic storage device, or any suitable combination of the foregoing.

To provide interaction with a user, the systems and techniques described herein may be implemented on a computer. The computer has: a display device, such as a cathode ray tube (CRT) or liquid crystal display (LCD) monitor, for displaying information to the user; and a keyboard and a pointing device (e.g., a mouse or trackball) through which the user can provide input to the computer. Other kinds of devices may also be used to provide interaction with the user; for example, the feedback provided to the user may be any form of sensory feedback (e.g., visual feedback, auditory feedback, or tactile feedback); and may be provided in any form, including acoustic input, speech input, or tactile input) to receive input from the user.

The systems and techniques described herein may be implemented in a computing system including back-end components (e.g., as a data server), or a computing system including middleware components (e.g., an application server), or a computing system including front-end components (e.g., a user's computer having a graphical user interface or web browser through which the user can interact with implementations of the systems and technologies described herein), or a computing system including any combination of such backend components, middleware components, or front-end components. The components of the system may be interconnected by any form or medium of digital data communication (e.g., a communications network). Examples of communication networks include: local area network (LAN), wide area network (WAN), and the Internet.

Computer systems may include clients and servers. Clients and servers are generally remote from each other and typically interact over a communications network. The relationship of client and server is created by computer programs running on corresponding computers and having a client-server relationship with each other. The server may be a cloud server, or a distributed system server, or a server combined with a blockchain.

It should be understood that various forms of the process shown above may be used, with steps reordered, added or deleted. For example, various steps described in the present disclosure may be executed in parallel, sequentially, or in a different order. As long as the desired results of the technical solution of the present disclosure can be achieved, there is no limitation here.

The above descriptions are merely specific implementation manners of the present disclosure, but the protection scope of the present disclosure is not limited thereto. It will be understood by those skilled in the art that various modifications, combinations, sub-combinations and substitutions may be made depending on design requirements and other factors. Any modifications, equivalent substitutions, improvements, etc. made within the spirit and principles of the present disclosure shall be included in the protection scope of the present disclosure.

What is claimed is:

1. A method for generating a radiation therapy plan, comprising:
acquiring a first radiation therapy plan for an object that is to be treated with radiation therapy in a first radiation therapy mode;
in response to a dose conversion operation on the first radiation therapy plan, acquiring a first effective dose field obtained by dose conversion of the first radiation therapy plan, wherein the first effective dose field is used to evaluate effect of radiation therapy based on the first radiation therapy plan on different radiation volumes in the object; and
generating a second radiation therapy plan for the object in a second radiation therapy mode using the first effective dose field as a basic dose field.

2. The method according to claim 1, wherein using the first effective dose field as the basic dose field, includes:
acquiring a dose field selection operation; and
among an original dose field of the first radiation therapy plan and the first effective dose field, using the first effective dose field as the basic dose field if it is determined that the first effective dose field is selected.

3. The method according to claim 2, wherein acquiring the dose field selection operation, includes:
evaluating whether the original dose field of the first radiation therapy plan and the first effective dose field satisfy a first prescription condition;
acquiring the dose field selection operation based on an evaluation result of the evaluating.

4. The method according to claim 3, wherein generating the second radiation therapy plan for the object in the second radiation therapy mode using the first effective dose field as the basic dose field, includes:
in a case where the first effective dose field satisfies the first prescription condition, generating the second radiation therapy plan for the object in the second radiation therapy mode using the first effective dose field as the basic dose field.

5. The method according to claim 3, further comprising: in a case where the first effective dose field does not satisfy the first prescription condition, after generating the second radiation therapy plan for the object in the second radiation therapy mode using the first effective dose field as the basic dose field,
determining a dose difference value of a target radiation volume in radiation volumes corresponding to the first radiation therapy plan, wherein the target radiation volume refers to a radiation volume not reached a first prescription dose of the first radiation therapy mode, and the dose difference value represents a difference value between an effective dose of the target radiation volume and the first prescription dose; and
increasing the effective dose of the target radiation volume corresponding to the second radiation therapy plan by a dose corresponding to the dose difference value, based on the dose difference value of the target radiation volume.

6. The method according to claim 1, wherein generating a second radiation therapy plan for the object in a second radiation therapy mode using the first effective dose field as a basic dose field, includes:
determining an objective function of the second radiation therapy mode based on a second prescription dose of the second radiation therapy mode; and
performing inverse optimization on the objective function using the first effective dose field as the basic dose field, to obtain the second radiation therapy plan.

7. The method according to claim 1, wherein in response to the dose conversion operation on the first radiation therapy plan, acquiring the first effective dose field obtained by dose conversion of the first radiation therapy plan, includes:
in response to the dose conversion operation on the first radiation therapy plan, determining an effective dose corresponding to each radiation volume corresponding to the first radiation therapy plan, based on a radiation dose of each radiation volume, a fractionated radiation dose of each radiation volume, and a radiosensitivity parameter corresponding to each radiation volume, to obtain the first effective dose field.

8. The method according to claim 1, further comprising: after generating the second radiation therapy plan for the object in the second radiation therapy mode using the first effective dose field as the basic dose field,
in response to the dose conversion operation on the second radiation therapy plan, acquiring a second effective dose field obtained by dose conversion of the second radiation therapy plan, wherein the second effective dose field is used to evaluate effect of radiation therapy based on the second radiation therapy plan on different radiation volumes in the object; and
evaluating whether the second radiation therapy plan satisfies a second prescription condition based on the second effective dose field.

9. The method according to claim 8, further comprising: after in response to the dose conversion operation on the second radiation therapy plan, acquiring the second effective dose field obtained by dose conversion of the second radiation therapy plan,
performing superposition processing on the first effective dose field and the second effective dose field to obtain a comprehensive effective dose field of the first radiation therapy plan and the second radiation therapy plan; and
evaluating whether comprehensive treatment effect of the first radiation therapy plan and the second radiation therapy plan satisfies a third prescription condition based on the comprehensive effective dose field.

10. A method for generating a radiation therapy plan, comprising:
acquiring a first radiation therapy plan for an object that is to be treated with radiation therapy in a first radiation therapy mode;
in response to a dose conversion operation on the first radiation therapy plan, acquiring a first effective dose field obtained by dose conversion of the first radiation therapy plan, wherein the first effective dose field is used to evaluate effect of radiation therapy based on the first radiation therapy plan on different radiation volumes in the object;
evaluating whether an original dose field of the first radiation therapy plan and the first effective dose field satisfy a first prescription condition; and
generating a second radiation therapy plan for the object in a second radiation therapy mode using one, satisfying the first prescription condition, of the original dose field and the first effective dose field as the basic dose field, based on an evaluation result of the evaluating.

11. The method according to claim 10, wherein in a case where the original dose field satisfies the first prescription condition, the second radiation therapy plan for the object in the second radiation therapy mode is generated using the original dose field as the basic dose field.

12. An electronic device, comprising:
at least one processor; and
a memory communicatively connected to the at least one processor, wherein the memory stores instructions executable by the at least one processor; and the instructions, when executed by the at least one processor, cause the at least one processor to perform the method according to claim 1.

13. An electronic device, comprising:
at least one processor; and
a memory communicatively connected to the at least one processor, wherein the memory stores instructions executable by the at least one processor; and the instructions, when executed by the at least one processor, cause the at least one processor to perform the method according to claim 10.

14. A non-transitory computer-readable storage medium, having stored computer instructions that, when run on an electronic device, cause the electronic device to perform the method according to claim 1.

15. A non-transitory computer-readable storage medium, having stored computer instructions that, when run on an electronic device, cause the electronic device to perform the method according to claim 10.
